(19) United States
(12) Patent Application Publication
(10) Pub. No.: US 2021/0338993 A1
(43) Pub. Date: Nov. 4, 2021

(54) Topical Medicine Applicator for Scalp

(71) Applicant: Thomas Jefferson University, Philadelphia, PA (US)

(72) Inventors: Elizabeth Rose Schoenberg, Bala Cynwyd, PA (US); Stefani Lynne Samuels, Ambler, PA (US); Vivek Koratkar Bilolikar, Maple Grove, MN (US); Jennifer Lauren Doto, Philadelphia, PA (US); Matthew James Sharayko, Harleysville, PA (US)

(21) Appl. No.: 17/286,539

(22) PCT Filed: Oct. 17, 2019
(86) PCT No.: PCT/US2019/056689

§ 371 Date: Apr. 19, 2021

Related U.S. Application Data
(60) Provisional application No. 62/747,035, filed on Oct. 17, 2018.

(51) Int. Cl. A61M 35/00

(52) U.S. Cl. CPC A61M 35/003; A61M 2210/06

(57) ABSTRACT

A medicine applicator includes a pressurized canister. The pressurized canister contains a medicine. The pressurized canister includes a nozzle. The nozzle includes a valve. The medicine applicator includes a handle, wherein, in response to a motion of the handle, the valve of the nozzle can be activated to release the medicine from the pressurized canister. The medicine applicator includes a distributor attached to a top portion of the nozzle. The medicine applicator includes a scalp contactor attached to the distributor.

### CROSS-REFERENCE TO RELATED APPLICATIONS

This application claims priority to U.S. provisional application No. 62/747,035 filed on Oct. 17, 2018 incorporated herein by reference in its entirety.

### FIELD OF THE DISCLOSURE

The present invention relates generally to topical medicine applicator for scalp. More specifically, the embodiments disclosed herein are designed to conveniently distribute topical medicine to scalp for treating any scalp related skin conditions, e.g., scalp psoriasis.

### BACKGROUND

Patients with scalp psoriasis have a hard time properly applying the medicine because usually scalp is covered by hair. Currently, a patient with scalp psoriasis needs to use both hands to apply the medicines. A patient needs to use one hand to keep the hairs in place to expose the intended area of scalp and use the other hand to apply the medicine. In most cases, the majority of the medicine applied is wasted on the hair. Further, the application process of using two hands is not convenient and is inefficient.

The embodiments disclosed herein provide a topical medicine applicator for scalp. The applicator can apply the medicine directly to scalp, avoiding waste of medicine on hairs. The applicator can be operated with single hand, providing a convenient way to apply medicine to the scalp compared to current solutions.

### SUMMARY

The present invention relates generally to topical medicine applicator for scalp. More specifically, the embodiments disclosed herein are designed to conveniently distribute topical medicine, e.g., foams, to scalp for treating any scalp related skin conditions, e.g., scalp psoriasis.

In one embodiment, a medicine applicator includes a pressurized canister. The pressurized canister contains a medicine. The pressurized canister includes a nozzle. The nozzle includes a valve. The medicine applicator includes a handle, wherein, in response to a motion of the handle, the valve of the nozzle can be activated to release the medicine from the pressurized canister. The medicine applicator includes a distributor attached to a top portion of the nozzle. The medicine applicator includes a scalp contactor attached to the distributor.

In one embodiment, a method to use the medicine applicator includes: pressing a gripping portion of the handle 405; activating a valve of a nozzle 410; releasing a medicine stored in a pressurized canister from an end of the nozzle 415; passing the medicine through an internal tunnel of a fitter of a distributor 420; passing the medicine through cavity disposed between a scalp contactor and the distributor 425; passing the medicine through a plurality of internal tunnels of fingers of the scalp contactor 430; and expelling the medicine from tips of the fingers 435.

In one embodiment, a medicine applicator includes a pressurized canister comprising a chamber in fluid communication with a nozzle, and a valve configured to control flow between the chamber and the nozzle; and a scalp contactor comprising a plurality of protrusions having a plurality of internal tunnels, the plurality of internal tunnels in fluid communication with the nozzle.

The foregoing has outlined rather broadly the features and technical advantages of the present invention such that the detailed description of the invention that follows may be better understood. Additional features and advantages of the invention will be described hereinafter that form the subject of the claims of the invention. It should be appreciated by those skilled in the art that the concepts and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the disclosed systems and methods, reference is now made to the following descriptions taken in conjunction with the accompanying drawings. Similar numbers refer to similar features.

FIG. 1 is an assembly of a scalp medicine applicator according to one embodiment of the disclosure.

FIG. 2 is an assembly of a scalp medicine applicator according to one embodiment of the disclosure.

FIG. 3 is an assembly of a scalp medicine applicator according to one embodiment of the disclosure.

FIG. 4 shows a method 400 for using the scalp medicine applicator according to one embodiment of the disclosure.

### DETAILED DESCRIPTION

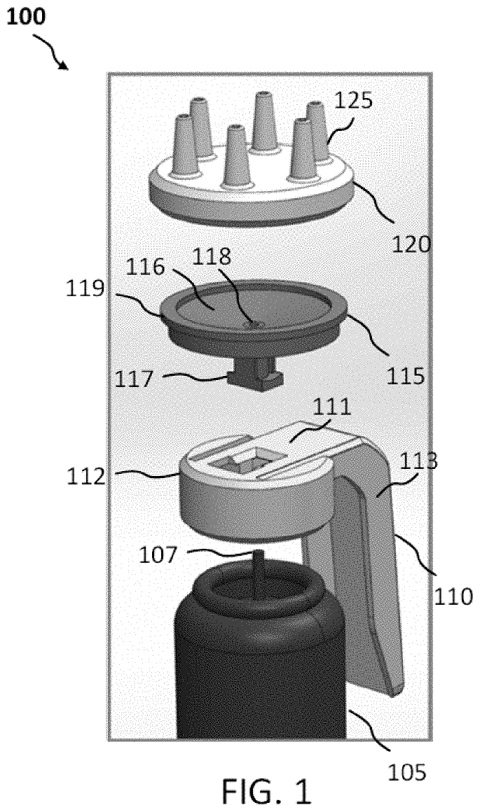

FIG. 1 is an assembly of a scalp medicine applicator 100 according to one embodiment of the disclosure. The applicator 100 includes a pressurized canister 105 containing medicine. In one embodiment, the medicine contained in the canister 105 can be clobetasol foam, clobetasol solution, mineral oil, general oil/petroleum based products, shampoos, salicylic acid, and combinations thereof. The canister 105 is in a cylindrical shape with an internal cavity for storing the medicine. The internal cavity is releasably sealed. The internal cavity can release the medicine through a controlled valve, e.g., the nozzle 107.

The canister 105 includes a nozzle 107 for releasing the medicine from the canister chamber 109 pressurized canister 105 via actuation of a valve 108. The nozzle 107 is an elongated protrusion disposed at the center on a top portion of the canister 105. The nozzle 107 includes a valve 108. In one embodiment, the valve 108 is opened when the nozzle 107 is pushed downward, releasing the medicine. In another embodiment, the valve 108 is opened when the nozzle is pushed sideways, e.g., tilted, releasing the medicine.

The applicator 100 includes a handle connector 112 and a handle 110. The handle connector 112 is in a shape of a cylinder having a portion carved out to accommodate the gripping portion 113 of the handle 110. The handle connector 112 includes an internal cavity. The internal cavity of the handle connector 112 receives at least a top portion of the pressurized canister and the nozzle 107. The handle connector 112 has a lower circular circumference. The lower circular circumference seals with a top ring portion of the pressurized canister 105.

The handle 110 has a horizontal portion 111 that is parallel to a top surface of the handle connector 110. The handle 110 has a gripping portion 113 that protrudes downward from a side of the horizontal portion 111. The horizontal portion 111 includes an opening that receives a fitter 117. The horizontal portion 111 is connected to the handle connector 112 at one side, opposite to the side connected to the gripping portion 113. When the gripping portion 113 is pressed by the patient, the horizontal portion 111 may either push the nozzle 107 downward or to the side, opening the valve of the nozzle 107 and releasing the medicine from the canister 105.

The applicator 100 includes a distributor 115. The distributor 115 includes a fitter 117, a concave structure 116, an opening 118, and a rim 119. The fitter 117 is a protrusion extending downward from the concave structure 116. The fitter 117 extends through the opening of the horizontal portion 111 and fits around the nozzle 107. The fitter 117 seals about the nozzle 107. The fitter 117 has an interval cavity that receives the nozzle 107. The internal cavity of the fitter 117 is fluidly connected to the opening 118 of the distributor 115.

The distributor 115 includes a concave structure 116. The opening 118 is disposed at the center of the concave structure 116. The rim 119 is disposed on a top surface of the concave structure 116 circumscribing around the top surface of the concave structure 116.

The rim 119 attaches to and seals around a bottom surface of a scalp contactor 120. The scalp contactor 120 has a base. The base is a disk with multiple openings. Each opening is attached to a finger 125. The base includes a groove configured to receive the rim 119, such that the distributor 115 seals with the scalp contactor 120, forming a cavity that is fluidly connected with the pressurized canister 105.

A finger 125 is an elongated protrusion extends from a top surface of the base. The finger 125 has an internal tunnel 126 extending therethrough is open at both ends. The finger 125 may have a height of 0.1 to 2 inches, preferably 0.5 to 1 inch.

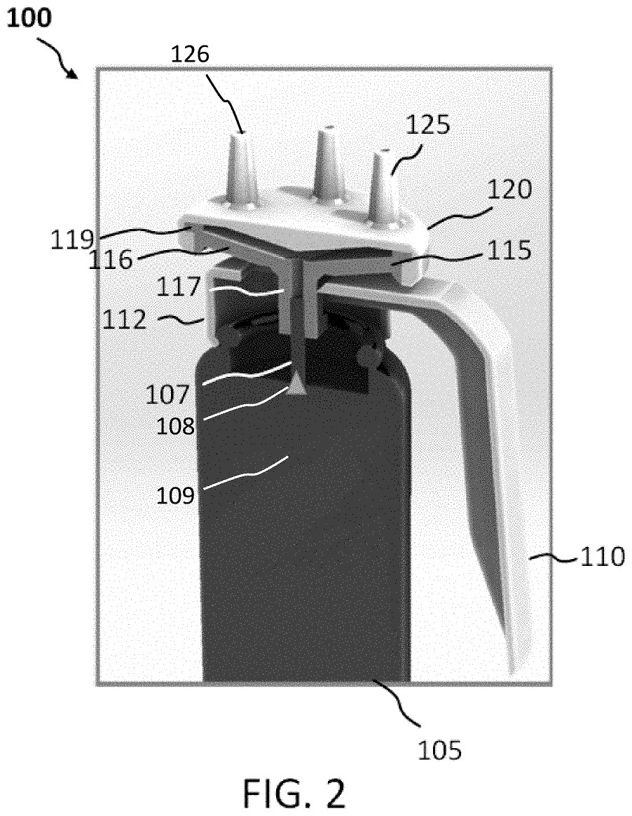

As shown in FIG. 2, the base of the scalp contactor 120 includes a groove configured to receive the rim 119 of the distributor 115, such that the distributor 115 seals with the scalp contactor 120, forming a cavity that is fluidly connected with the pressurized canister 105.

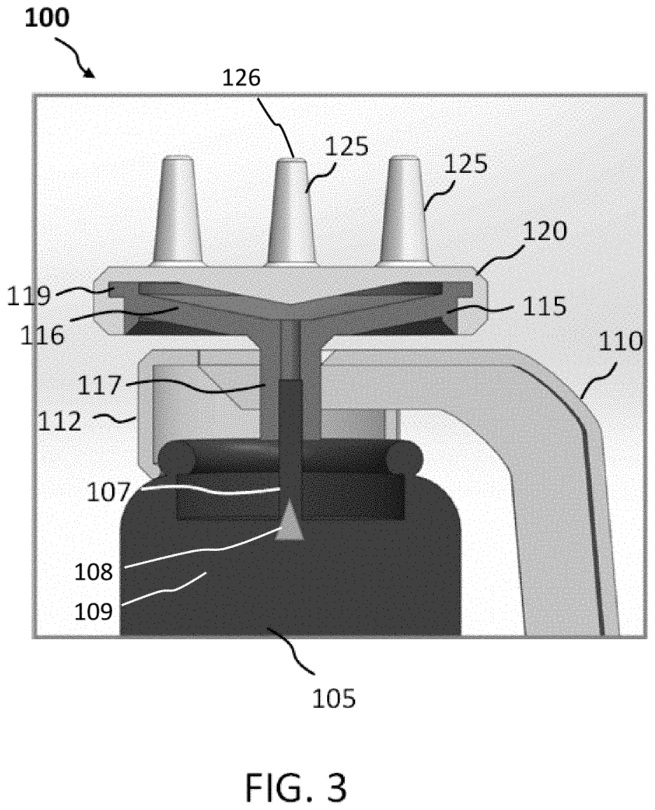

When the valve of the nozzle 107 is being released, the medicine from the canister 105 is pushed out. The medicine flows through the internal tunnel of the fitter 117, filling the cavity formed between the distributor 115 and the scalp contactor 120, then through the internal tunnels of the fingers 125, and eventually the medicine is expelled from the tip of the fingers. FIG. 3 shows a sectional view of the assembly of the scalp medicine applicator 100.

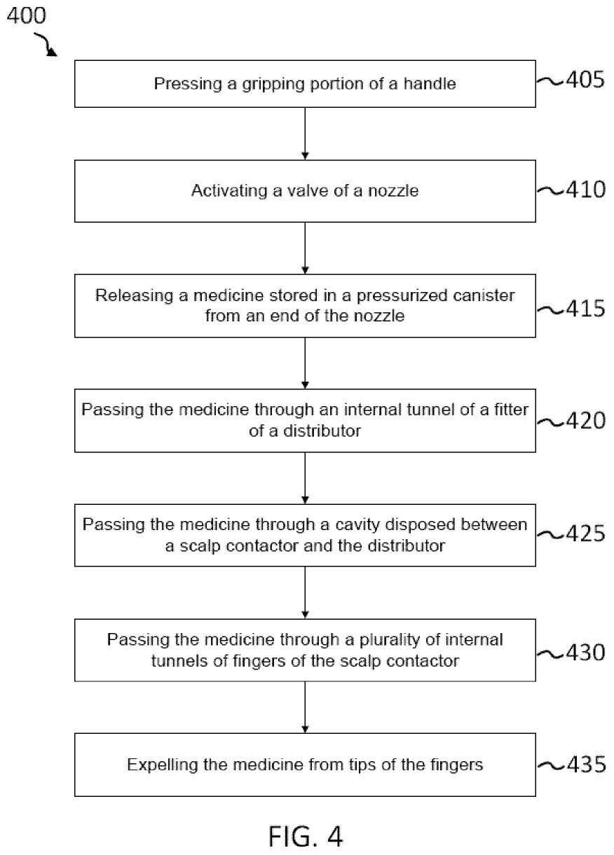

FIG. 4 shows a method 400 for using the scalp medicine applicator 100. The method includes: pressing a gripping portion of the handle 405; activating a valve of a nozzle 410; releasing a medicine stored in a pressurized canister from an end of the nozzle 415; passing the medicine through an internal tunnel of a fitter of a distributor 420; passing the medicine through cavity disposed between a scalp contactor and the distributor 425; passing the medicine through a plurality of internal tunnels of fingers of the scalp contactor 430; and expelling the medicine from tips of the fingers 435.

The method 400 may further including releasing the medicine on a topical surface of a scalp. The method 400 may further including positioning the tips of the fingers toward the scalp.

In the above disclosed embodiments, similar terms means similar elements. Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present invention, disclosure, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A medicine applicator, comprising
a pressurized canister, the pressurized canister containing a medicine, the pressurized canister includes a nozzle, the nozzle includes a valve;
a handle, wherein, in response to a motion of the handle, the valve of the nozzle can be activated to release the medicine from the pressurized canister;
a distributor attached to a top portion of the nozzle; and
a scalp contactor attached to the distributor.

2. The medicine applicator according to claim 1, wherein
the handle includes a handle connector, a horizontal portion, and a gripping portion, the handle connector fits around a top portion of the pressurized canister, the horizontal portion has a top surface parallel to a top surface of the handle connector, the gripping portion protrudes downward from a side of the horizontal portion.

3. The medicine applicator according to claim 2, wherein
the horizontal portion includes an opening allowing a fitter of the distributor to pass through.

4. The medicine applicator according to claim 1, wherein
the distributor includes a fitter, the fitter passes through the handle, the fitter attaches to a top portion of the nozzle, the fitter is fluidly connected to the nozzle.

5. The medicine applicator according to claim 4, wherein
the distributor includes a concave structure, the distributor includes a rim disposed on top of the concave structure.

6. The medicine applicator according to claim 5, wherein the rim seals with a lower portion of the scalp applicator forming a cavity between the distributor and the applicator, the cavity is fluidly connected to the nozzle.

7. The medicine applicator according to claim 1, wherein the scalp contactor includes a base and a plurality of fingers.

8. The medicine applicator according to claim 7, wherein the base includes a same number of openings, each opening is connected to a finger.

9. The medicine applicator according to claim 8, wherein each finger includes an internal tunnel that is fluidly connected to the nozzle, each finger has an opening at a tip for expelling the medicine.

10. The medicine applicator according to claim 1, wherein the scalp contactor is in contact with a scalp of a patient.

11. A method for applying medicine to a scalp using a medicine applicator, comprising
holding the medicine applicator, the medicine applicator further including
a pressurized canister, the pressurized canister containing a medicine, the pressurized canister includes a nozzle, the nozzle includes a valve;
a handle connected to the nozzle;
a distributor attached to a top portion of the nozzle; and
a scalp contactor attached to the distributor;
pressing a gripping portion of the handle;
activating the valve of the nozzle; and
releasing the medicine from the pressurized canister.

12. The method according to claim 11, further comprising
passing the medicine through an internal tunnel of a fitter of the distributor.

13. The method according to claim 11, further comprising
passing the medicine through a cavity disposed between the scalp contactor and the distributor.

14. The method according to claim 11, further comprising
passing the medicine through a plurality of internal tunnels of fingers of the scalp contactor.

15. The method according to claim 11, further comprising
expelling the medicine from tips of the fingers.

16. The method according to claim 11, further comprising
contacting the fingers with a scalp.

17. A medicine applicator, comprising:
a pressurized canister comprising a chamber in fluid communication with a nozzle, and a valve configured to control flow between the chamber and the nozzle; and
a scalp contactor comprising a plurality of protrusions having a plurality of internal tunnels, the plurality of internal tunnels in fluid communication with the nozzle.
